(19) United States
(12) Patent Application Publication
(10) Pub. No.: US 2011/0284036 A1
(43) Pub. Date: Nov. 24, 2011

(54) CONTAINER HAVING MEANS FOR SANITISING AND PROTECTING A TOOTHBRUSH

(75) Inventor: Richard Skoulding, Suffolk (GB)

(73) Assignee: Richard SKOULDING, Suffolk (GB)

(21) Appl. No.: 12/812,286

(22) PCT Filed: Jan. 11, 2009
(86) PCT No.: PCT/GB2009/000054

§ 371 Date: Mar. 7, 2011

(30) Foreign Application Priority Data
Jan. 11, 2008 (GB) 0800449.1

(51) Int. Cl. A47K 1/09

(52) U.S. Cl. 134/32; 206/361

(57) ABSTRACT

A toothbrush holder (1) comprises a container (2) defining a hollow body so sized and shaped as to accommodate a toothbrush. At one end of the body there is an open mouth into which a toothbrush may be placed and a closing cap for sealing the holder so that sanitising liquid held within the holder does not leak in transit. A platform element or cupped shuttle (5) receives the teeth-cleansing bristles carrying head of a toothbrush the shuttle being manually urged towards the base end of the cylindrical body against a biasing spring (4) to immerse the bristles (B) thereof into an amount of sanitising liquid (8). A closing cap is optionally provided at the base end of the holder to facilitate draining, cleaning, assembly and disassembly of the holder. The or each cap may be threaded for engagement to the body of the holder or may be of the push fit and twist type.

### FIELD OF THE INVENTION

The present invention relates to a container for a toothbrush and more particularly to a toothbrush holder that minimises the microbial and bacterial growth on the bristles and head of a toothbrush. The invention further relates to a travel container for a toothbrush and a method of cleaning the head of a toothbrush.

### BACKGROUND TO THE INVENTION

It is a widely acknowledged but poorly appreciated fact that the bathroom is a poor environment in which to store a toothbrush. The primary source of potential contamination is the toilet which can eject an aerosol spray of microbe-laden water from within the toilet bowl on each flush. Additionally, food particles often remain within the bristle tufts even after a toothbrush is rinsed and this provides a fertile ground for bacteria.

Most often toothbrushes are stored in a tumbler or in a toothbrush holder comprising one or more simple loops made of a metal or plastics material usually attached to a wall in a bathroom adjacent a sink. Unfortunately, the toothbrush is located in an open environment and/or resting on a surface unlikely to be regularly cleaned.

This exposure allows a wide variety of bacteria, microbes, viruses and other matter to be held within the bristles of the toothbrush, significantly increasing the potential risk of infection and ill-health for the user.

A common solution to the above problem is to store the toothbrush inverted in a tumbler or mug keeping the head of the toothbrush immersed in a sanitising liquid, most commonly, an antiseptic liquid or a readily available mouthwash. This means of addressing the problem also has its inherent disadvantages. Firstly, the top of a tumbler is open and therefore the sanitising liquid is exposed to the same microbes and bacteria as the toothbrush head was previously and it is likely that one or more toothbrushes will be used in the same container and more foreign material will be introduced to the liquid on each occasion. Eventually, the sanitising liquid will lose its efficacy and will itself become a breeding ground for bacteria.

The term “sanitising liquid” as used herein is not intended to be limiting and it will be appreciated by the skilled reader that any suitable liquid can be used; including salt water and effervescent tablets immersed in water.

The prior art has a number of proposed solutions to the above problems, most commonly arrangements for enclosing at least the head of the or each toothbrush so as not to be exposed to the surrounding environment. Exemplary of the prior art in this respect is International Patent Publication No WO2005/016100 to FRETWELL and SCHRAMM which discloses a toothbrush holder for enclosing the head of a toothbrush within a container having a closing lid which sealingly surrounds the shank of the toothbrush and retains a disinfecting or sterilising liquid within the container. There are also provided standalone individual containers for receiving a toothbrush and standing it inverted in a volume of antiseptic liquid. Few of these arrangements allow for the toothbrush and holder to be packed for travel. An example of the relevant prior art is that disclosed in International Patent Publication No. WO2004/062448 to COCHRAN and COCHRAN which describes a conical self-supporting container having a lid element which engages the shank or handle of a toothbrush and is profiled to sealingly fit the mouth aperture of the container. Particularly, none of the prior art arrangements allow for rinsing or cleaning of the toothbrush in situ or the removal of excess antiseptic liquid before the toothbrush is withdrawn from its container.

In French Patent Publication No. 2 477 878 to GODEUX, there is disclosed a toothbrush sanitising apparatus comprising a cylindrical container within which there is provided a vertically moveable compartment which is biased towards the open mouth of the container by a spring. The compartment has a calibrated aperture in the base thereof to retain a sterilising solution around the head of a toothbrush for a predetermined interval before the solution drains through the aperture. It is noted that the apparatus is configured to retain the toothbrush head with the sterilising solution only for as long as it takes for the solution to drain through the aperture and even then one end of the brush head will be immersed longer than the other meaning that only the free end of the toothbrush head gains maximum benefit of the invention. It is further observed that there is no rinsing action provided, thereby leaving debris within the brush bristles.

U.S. Pat. No. 5,950,648 to SKROCKI discloses an apparatus for cleaning paint application brushes for the art and fingernail industries. The apparatus comprises an elongate cylindrical container and a plinth or pier against which a support collar abuts. The collar is secured by interference fit to the shaft or handle of the brush and holds the head of the brush in a central hollow of the plinth or pier so that the head of the brush may be immersed in a cleaning fluid while preventing the cleaning fluid making contact with the brush handle. It is noted that the plinth or pier, which may be formed from spring coils, is provided only as a stop and a support structure for the brush and is statically positioned within the container. Pushing down on the brush handle merely moves the collar along the handle.

It is an object of the present invention to alleviate the above disadvantages and to provide a toothbrush container which can be sealed for storage between uses or for travel.

It is also an object of the present invention to provide an improved toothbrush container within which a sanitising or antiseptic liquid can be vigorously washed through the bristles and subsequently excess liquid removed from the bristles as the toothbrush is withdrawn from the container for use.

It is a yet further object of the invention to provide a method of maintaining a toothbrush in a substantially hygienic condition.

### SUMMARY OF THE INVENTION

Accordingly, the present invention provides a toothbrush holder comprising:
a substantially cylindrical hollow body so sized and shaped as to accommodate a toothbrush fully and defining at one end an open mouth for placing a toothbrush within the holder;
a closing cap which is sealingly engageable to the open mouth of the holder; and
a platform element slideably moveable within the cylindrical body and biased from a base end thereof towards the open mouth end, the platform element having at least one aperture therein to facilitate the substantially unrestricted flow of a sanitising liquid therethrough.

Conveniently, the platform element is biased by a spring coil located or secured between the base end of the cylinder and the platform element to facilitate a range of axial movement of the platform element within the cylindrical body equal to or greater than the length of a toothbrush head.

This arrangement allows sanitising liquid to be jetted around the bristles of a toothbrush when downward pressure is reciprocally applied against the bias of the spring.

Ideally, the platform element includes side walls to constrain movement thereof within the body and to engage the head of a toothbrush. The side walls ensure the platform element does not twist within the body under the influence of the biasing spring or a toothbrush being forced within the body and constrains the motion of the platform element to a purely axial movement.

In one arrangement, the toothbrush holder is provided with a closing cap at the base end of the cylindrical body, the cap being sealingly engageable thereto for facilitating the draining and cleaning of the holder.

The second closing cap optionally includes a flared base integrally formed therewith so as to enable the holder to stand upright stably. With a flared base, the toothbrush holder of the invention can be stored on any suitable flat surface and remains a convenient size and shape for travel purposes.

In an alternative arrangement, the flared base is integrally formed with the body of the holder.

In a further optional construction, a collar adapted to fit securely to the outer diameter of the cylindrical body is removable for storage or transportation and is positioned at the base end of the holder to provide a stabilising foot.

In yet another alternative arrangement, a flared base may be provided separately to the holder and adapted to receive the base end of the cylindrical body of one or more toothbrush holders.

The second closing cap or base of the hollow cylindrical body of the holder optionally includes a compartment for toothpaste (powder), floss or tablets, for example.

In the above arrangements, both the open mouth region and the base end region of the cylindrical body may be threaded so that correspondingly threaded closing caps are used to sealingly close the holder in use to prevent the escape of sterilising or disinfecting liquid during transit.

In an alternative arrangement, the or each closing cap engages the cylindrical body using a push fit and twist lock arrangement. With the push fit and twist lock arrangement, whereby corresponding keyed portions are provided in a cap and end region of the body, rinsing of the toothbrush is automatically achieved when a toothbrush is placed within the cylinder. Closing the lid pushes the tip of the toothbrush head against the platform element and pushes it downwardly towards the base end thereof. In this arrangement, where sterilising or antiseptic liquid is provided in the cylinder up to the level of the platform element, closure of the lid forces the toothbrush downwardly and into the liquid.

In one aspect of the invention, there is provided a toothbrush container comprising:
a substantially cylindrical tube having a base end and an open end, the open end having lid engagement means for sealingly securing a lid element thereto;
a platform element slidably moveable within the tube but constrained in its movement by a biasing spring connected to the platform element at one end and the base end of the tube at the other, and
a liquid barrier element disposed within the length of the tube.

The liquid barrier element acts to attenuate liquid flow towards the open end of the tube should the tube be knocked over or inverted during travel. Additionally, the liquid barrier element acts on the toothbrush bristles to remove excess sanitising liquid therefrom as the toothbrush is extracted from the tube prior to use.

In use, a measured amount of an antiseptic or sterilising liquid is poured into the tube to a predetermined level, the level being such that when a toothbrush is placed into the tube and depressed, the liquid covers the entire toothbrush head.

Ideally, a marked graduation is provided on the wall of the cylinder to indicate the level of liquid required.

Conveniently, the cylindrical body is made from a transparent plastics material.

To clean the toothbrush head thoroughly, the toothbrush is placed within the tube so that the head thereof rests on or within the platform element. The handle of the toothbrush protrudes from the mouth of the cylinder, which can be held in the hand, and the toothbrush depressed against the biasing action of the spring, in a reciprocating motion using the thumb. In doing so, the antiseptic liquid is forced through the apertures in the platform in an agitated motion, circulating about and through the bristles of the toothbrush head. Additionally, on each stroke the head is totally immersed in the liquid. It will further be appreciated that when the closing cap is in position, the toothbrush head is totally immersed in the liquid.

In normal storage, the lid of the container depresses the toothbrush handle (shank) and urges the toothbrush head and the platform element against the action of the biasing spring into the region at the closed end of the cylinder to be immersed and retained in the antiseptic liquid.

When the toothbrush is required for use, the lid is opened and the toothbrush handle is presented under the action of the biasing spring urging the platform element towards the open mouth of, the container and pushing the toothbrush head. Disposed within the cylinder between the platform element and the open mouth of the tube, there is provided a liquid barrier element to prevent the passage of excess liquid towards the open mouth. In a first mode, the liquid barrier acts to impede the flow of liquid out of the cylinder where the cylinder is accidentally inverted. In a second mode, the liquid barrier acts against the bristles of the toothbrush as it is retracted from the cylinder, thereby removing excess liquid from the bristles.

### BRIEF DESCRIPTION OF THE DRAWINGS

The invention will now be described more particularly with reference to the accompanying drawings which show, by way of example only, one embodiment of toothbrush container in accordance with the invention. In the drawings:

FIG. 1 is a side elevation of a toothbrush container with a toothbrush resting inside;

FIG. 2 is a side elevation of the first embodiment of toothbrush container in which the toothbrush has been forced downwardly into antiseptic liquid by the closure of the lid; and

FIGS. 3a and 3b are a sectional side elevation and a plan view of a platform element.

### DETAILED DESCRIPTION OF THE PREFERRED EMBODIMENT

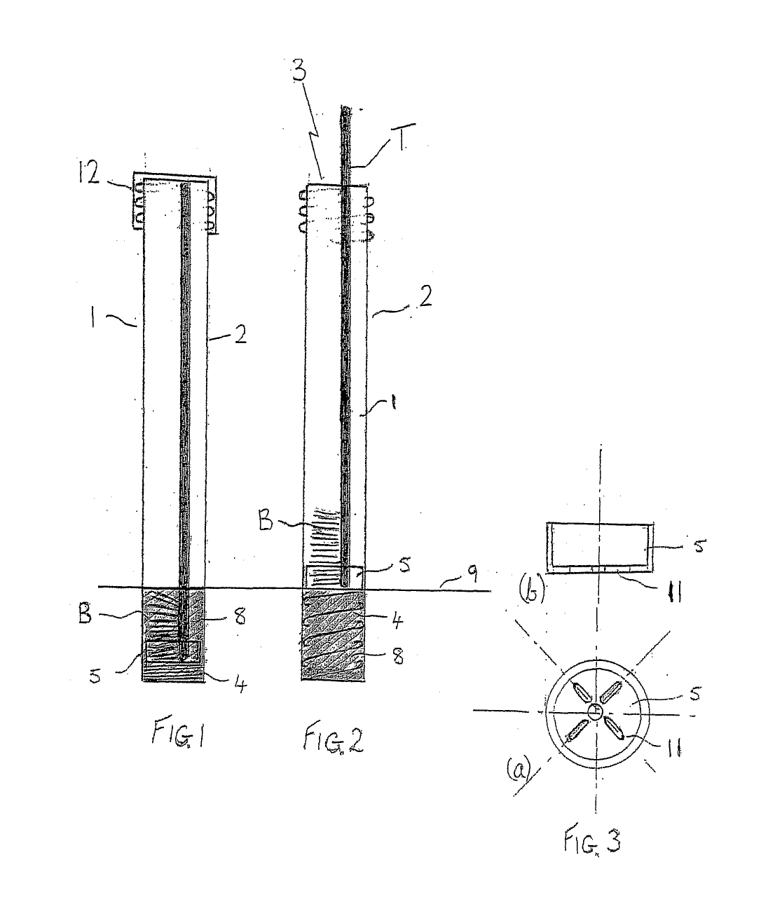

Referring to the drawings and initially to FIGS. 1 and 2, a toothbrush travel container 1 comprises a cylindrical hollow body 2 having an open mouth 3 at one end and closed end where a compression spring 4 is loosely positioned at the bottom of the container 1. Over the compression spring 4, a cupped shuttle 5 defining the platform element is positioned. The cupped shuttle 5 is slideable within the inner wall of the hollow body 2 and rests on top of the spring. When the spring 4 is relaxed, that is to say without compression loading, the cupped shuttle is in a raised position, as shown in FIG. 2. A sanitizing liquid 8, typically a mouthwash or, alternatively, a specifically formulated antiseptic liquid is poured into the open mouth 3 of the container 1. The amount of liquid 8 used will ideally just cover the spring 4 and be to the bottom of the cupped shuttle 5 and a graduation mark (not shown) is optionally provided to indicate the recommended level 9 for the liquid 8 on the hollow body 2. A toothbrush T can be introduced into the container 1 and will be supported within the cupped shuttle and disposed above the liquid 8 by the biasing resistance of the spring 4. This resistance is overcome when the top of the toothbrush T is pushed downwardly, which causes the cupped shuttle 5 to displace the liquid 8 and force the liquid through holes 11 provided in the cupped shuttle. The spring 4 compresses as a result of the handle of the toothbrush T being pushed downwardly. Bristles B on the head of the toothbrush are thus immersed in the sanitising or antiseptic liquid 8. When the toothbrush handle is released, the head of the toothbrush T is carried by the shuttle as the spring 4 reverts to its relaxed state and the bristles B are lifted from the liquid 8 or at least substantially so.

In this position liquid can be allowed to drain from the bristles under gravity, however, as a lot of additional sanitising liquid 8 can be retained in the toothbrush bristles B, a liquid barrier element (not shown) defining a constriction within the cylinder is provided. This constriction may take the form of a simple annular collar, wide enough to allow passage of the toothbrush head but sufficiently narrow to engage and deflect the bristles, so that as the bristles pass over the collar, liquid is firstly compressed from successive tufts of bristles as they are deflected in one direction and then flicked against the inner wall of the hollow body 2 as the bristles regain their relaxed state having passed the collar.

To clean the toothbrush head thoroughly, the portion of the toothbrush handle protruding from the open mouth of the container can be pushed downwardly against the cupped shuttle and spring to immerse the bristles in the sanitising liquid. Releasing the handle allows the bristles to rise out of the liquid and this action can be repeated rapidly several times to effectively clean the toothbrush head. With selected sanitising liquids, particularly ordinary mouthwash, the rapid cleansing movement produces a foaming action preferred by many users. The cleaning action is carried out by the liquid being forceably moved upwardly through the holes 11 in the cupped shuttle 5. Accordingly, the number of holes provided in the base of the shuttle 5 is selected to allow rapid transfer of the sanitising liquid and to aid a foaming action. A cap 12 can be located over the top of the toothbrush handle and screwed down onto a threaded portion 14 of the container 1 to seal the liquid within the body 2 of the container 1. When the cap 12 is secured the toothbrush bristles B are immersed in the liquid to be sanitized. When the cap 12 is removed in order to use the toothbrush T, the spring 4 relaxes and causes the cupped shuttle 5 to rise out of the liquid which will also lift the bristles B out of the liquid and the free end of the handle protrudes from the open mouth of the cylindrical body 2 to allow a user to remove the toothbrush T. The liquid drains from the shuttle cup 5 through the holes 11 and surplus liquid which is in the bristles will also drain through the same holes. Surplus sanitising liquid may be removed by the liquid barrier described hereinabove. The toothbrush T can then be removed from the container 1 and is free for use.

In an alternative embodiment (not shown), the threaded portion 14 is replaced by detents so sized and shaped as to engage a push-fit and twist lock lid arrangement. Although not illustrated, the toothbrush holder may include a second closing cap disposed at the base end of the cylindrical body and sealingly engageable therewith. This facilitates removal of the cupped shuttle and the biasing spring for cleaning and also for more regular draining of the sanitising liquid. The closing cap may include a flared base so as to allow the holder to stand upright more stably. In an alternative arrangement a base element may be provided separately to the holder and adapted to receive a plurality of holders.

In a yet further unillustrated embodiment, the base end of the cylindrical body is provided with a removable collar or cup of greater diameter than the body, which can be removed for travel, allowing the holder to be put in a pocket or handbag.

Due to the sealing action of the cap, the toothbrush is protected from airborne microbes and flying or crawling insects. Additionally, by providing reliable sealing and preventing leakage of the sanitising liquid, the toothbrush holder is easily transportable, with toothbrush and liquid in situ.

It will be appreciated by the skilled addressee that the liquid barrier element and the configuration of the platform element may be altered without detracting from the preferred construction of the invention described herein.

It will also be appreciated that certain terms as used herein are not intended to be limiting and are representative only of a wider use or application.

It will of course be understood that the invention is not limited to the specific details described herein, which are given by way of example only, and that various modifications and alterations are possible within the scope of the appended claims.

1. A toothbrush holder comprising:
a substantially cylindrical hollow body so sized and shaped as to accommodate a toothbrush fully and defining at one end an open mouth for placing a toothbrush within the holder;
a closing cap which is sealingly engageable to the open mouth of the holder; and
a platform element slideably moveable within the cylindrical body and biased from a base end thereof towards the open mouth end, the platform element having at least one aperture therein to facilitate the substantially unrestricted flow of a sanitising liquid therethrough.

2. A toothbrush holder as claimed in claim 1, in which the platform element is biased by a spring coil located between the base end of the cylinder and the platform element to facilitate a range of axial movement of the platform element within the cylindrical body equal to or greater than the length of a toothbrush head.

3. A toothbrush holder as claimed in claim 1, in which the platform element includes side walls to constrain movement thereof within the body and to engage the head of a toothbrush.

4. A toothbrush holder as claimed in claim 3, in which the side walls are so sized and shaped as to ensure the platform element does not twist under the influence of the biasing spring or under the influence of a toothbrush being forced into the body and constrains the motion of the platform element to a purely axial movement.

5. A toothbrush holder as claimed in claim 1, in which there is provided at the base end of the cylindrical body a second closing cap sealingly engageable thereto for facilitating draining and cleaning of the holder.

6. A toothbrush holder as claimed in claim 5, in which the second closing cap of the holder includes a compartment for toothpaste (powder), floss, tablets and the like.

7. A toothbrush holder as claimed in claim 5, in which the second closing cap includes a flared base integral therewith so as to enable the holder to stand upright stably.

8. A toothbrush holder as claimed in claim 1, in which a flared base is integrally formed with the body of the holder.

9. A toothbrush holder as claimed in claim 8, in which the base of the hollow cylindrical body of the holder includes a compartment for toothpaste (powder), floss, tablets and the like.

10. A toothbrush holder as claimed in claim 1, in which a collar adapted to fit securely to the outer diameter of the cylindrical body is removable for storage or transportation and is positioned at the base end of the holder to provide a stabilising foot.

11. A toothbrush holder as claimed in claim 1, in which a flared base is provided separately to the holder and adapted to receive the base end of the cylindrical body of one or more toothbrush holders.

12. A toothbrush holder as claimed in claim 1, in which the, or each, closing cap engages the cylindrical body using a push fit and twist lock arrangement.

13. A toothbrush holder as claimed in claim 1, in which there is disposed a liquid barrier element within the cylindrical body of the holder.

14. A toothbrush holder as claimed in claim 13, in which the liquid barrier element is so sized and shaped as to prevent the passage of excess liquid towards the open mouth and in which, in a first mode, the liquid barrier acts to impede the flow of liquid out of the cylindrical body and, in a second mode, the liquid barrier acts against the bristles of a toothbrush as it is retracted from the body, thereby removing excess liquid from the bristles.

15. A toothbrush holder as claimed in claim 1, in which a measured amount of a sanitising liquid is poured into the holder to a predetermined level, the level being such that when a toothbrush is placed into the holder and depressed, the liquid covers the entire toothbrush head.

16. A toothbrush holder as claimed in claim 1, in which a marked graduation is provided on the cylindrical body to indicate the level of liquid required.

17. A toothbrush holder as claimed in claim 1, in which the container is made from a transparent plastics material.

18. A method of cleaning a toothbrush, the method including placing a toothbrush within a toothbrush holder of the type having a hollow cylindrical body and a platform element slidably and axially moveable within the body, so that the head of the toothbrush rests on or within the platform element, the handle of the toothbrush protruding from the mouth of the cylinder, which can be held in the hand, and depressing repeatedly the toothbrush against the return biasing action of a spring to effect a rinsing action.

19. A method of cleaning a toothbrush as claimed in claim 18, in which the method includes using a thumb, in a reciprocating motion on the exposed handle of the toothbrush, whereby in doing so, a sanitising liquid is forced through apertures in the platform in an agitated motion, circulating about and through the bristles of the toothbrush head.

20. A method of cleaning a toothbrush as claimed in claim 18, in which a measured amount of a sanitising liquid is poured into the holder to a predetermined level, the level being such that when a toothbrush is placed into the holder and depressed, the liquid covers the entire toothbrush head.

21. (canceled)

22. (canceled)
